(19) United States
(12) Patent Application Publication
(10) Pub. No.: US 2017/0340356 A1
(43) Pub. Date: Nov. 30, 2017

(54) VENTED TIP FOR FIXED-ABRASIVE MICRODERMABRASION WITH VACUUM-ASSISTED DEBRIS REMOVAL

(71) Applicant: PRESSER DIRECT, LLC, Los Angeles, CA (US)

(72) Inventors: Mark Presser, Los Angeles, CA (US); Daniel S.C. Kwok, Kowloon (HK)

(21) Appl. No.: 15/605,744

(22) Filed: May 25, 2017

Related U.S. Application Data
(60) Provisional application No. 62/341,361, filed on May 25, 2016.

(51) Int. Cl. A61B 17/54; A61B 90/00; A61B 17/00; A61B 17/32

(52) U.S. Cl. CPC A61B 17/54; A61B 17/545; A61B 2017/320004; A61B 2090/036; A61B 2090/0807; A61B 2217/005; A61B 2017/00761

(57) ABSTRACT

A vented microdermabrasion tip includes a disk-shaped abrasive component coated or configured with a microdermabrasive surface and fixed to a tubular sleeve by a set of annular mounts. The sleeve surrounds an outer periphery of the abrasive component, so that a gap is provided between the abrasive component and an internal air passage through the sleeve. A distal surface of the abrasive component is continuous, free of holes and coated with an abrasive material or formed with an abrasive surface. The distal surface extends beyond the distal end of the sleeve. An end of the mount opposite to the abrasive component is configured for coupling to a channel of a suction apparatus leading to an inlet of an air pump.

### CROSS REFERENCE TO RELATED APPLICATION

The present application claims priority to U.S. provisional application Ser. No. 62/341,361 filed May 25, 2016, which application is incorporated herein by reference.

### FIELD

The present disclosure relates to a vented fixed-abrasive microdermabrasion tip, and method of use thereof.

### BACKGROUND

Various portable microdermabrasion tools are known in the art, characterized by a microdermabrasion tip and a suction motor. The microdermabrasion tip may be powered by a rotary motor, or may be fixed. Suction through the tip may be used to draw in removed skin that is captured in an air filter. An air pump is used to supply suction. The suction may also draw in soft skin, choking off the flow of air through the tip and pulling the skin against the abrasive surface of the tip. For some individuals, the resulting abrasive effect may be greater than desired. Also, the choking off of air flow may alternate with times when the flow is not choked, causing variations in pulling force and reducing effectiveness of the suction for debris removal. This variation may make it difficult for the user to maintain a consistent abrasive effect over different areas of skin, or to control the level of microdermabrasion applied.

Notwithstanding the advantages of prior microdermabrasion tips and devices, there is a need for an improved microdermabrasion tip that can be used with existing suction devices and that overcomes the limitations explained above. The present tip fulfills this need and provides further related advantages, as described below. Reference will be made to the appended sheets of drawings which will first be described briefly.

### SUMMARY

A vented microdermabrasion tip includes a disk-shaped abrasive component coated or configured with a microdermabrasive surface and fixed to a tubular sleeve by a set of annular mounts. The sleeve may surround an outer periphery of the abrasive component, so that a gap is provided between the abrasive component and an internal air passage through the sleeve. A distal surface of the abrasive component may be continuous, free of holes and coated with an abrasive material or formed with an abrasive surface. The distal surface may extend beyond the distal end of the sleeve. A coated abrasive material may be, or may include, a diamond material or metal oxide. An end of the mount opposite to the abrasive component may be configured for coupling to a channel of a suction apparatus leading to an inlet of an air pump.

An ergonomically contoured handle of the suction apparatus holding an air pump for drawing air through the tip may be divided into at least two articulating sections separated by an annular cam ring, wherein each of the at least two articulating sections are coupled to one another and rotatable around a longitudinal axis of a longer one of the at least two articulating sections, whereby a distal end of the handle is displaced from the longitudinal axis. The device further includes an air pump disposed in at least one of the at least two articulating sections configured to draw air through the tip. The longer one of the at least two articulating sections may be configured for gripping.

The suction apparatus may further include a filter interposed between the microdermabrasion tip and the inlet of the air pump. The air pump may be configured to draw at least 3 liters per minute through the channel. An inlet of the air pump may be fluidly coupled to the channel of the microdermabrasion tip by a flexible hose. An electric power control circuit disposed in one of the at least two articulating sections may be configured to drive the air pump to draw air through treatment head disposed at the distal end of the handle at varying speeds.

The mount may include a cylindrical outer surface sized to slip inside a cylindrical inner surface of the microdermabrasion tip, providing a microdermabrasion assembly. The cylindrical outer surface may include a double O-ring seal. A second microdermabrasion tip may be provided, interchangeable with the microdermabrasion tip on the mount.

A more complete understanding of the microdermabrasion tip and its method of use will be afforded to those skilled in the art, as well as a realization of additional advantages and objects thereof, by a consideration of the following detailed description of the preferred embodiment. Reference will be made to the appended sheets of drawings, which will first be described briefly.

### BRIEF DESCRIPTION OF THE DRAWINGS

In the figures described below, like reference numerals are used to indicate like elements appearing in one or more of the figures.

FIG. 1 is a perspective view of a vented microdermabrasion tip with a fixed abrasive surface.

FIG. 2 is a side view of the microdermabrasion tip of FIG. 1.

FIG. 3 is a section view of the microdermabrasion tip of FIG. 1.

FIG. 4 is an exploded perspective view of the microdermabrasion tip of FIG. 1.

FIG. 5 is a top view of the microdermabrasion tip of FIG. 1.

FIG. 6 is a front view of an assembly including the microdermabrasion tip of FIG. 1 assembled to a suction apparatus.

FIG. 7 is a rear view of the assembly of FIG. 6.

FIG. 8 is a left side view of the assembly of FIG. 6.

FIG. 9 is a right side view of the assembly of FIG. 6.

FIG. 10 is a top plan view of the assembly of FIG. 6.

FIG. 11 is a bottom view of the assembly of FIG. 6.

FIG. 12 is a side section view of the assembly of FIG. 6.

FIG. 13 is an exploded perspective view of the assembly of FIG. 6.

FIG. 14 is a side schematic view showing aspects of an alternative embodiment of a vented microdermabrasion tip with a fixed abrasive surface.

FIG. 15 is a side schematic view showing aspects of an alternative embodiment of a microdermabrasion tip with a fixed, resilient abrasive surface.

### DETAILED DESCRIPTION

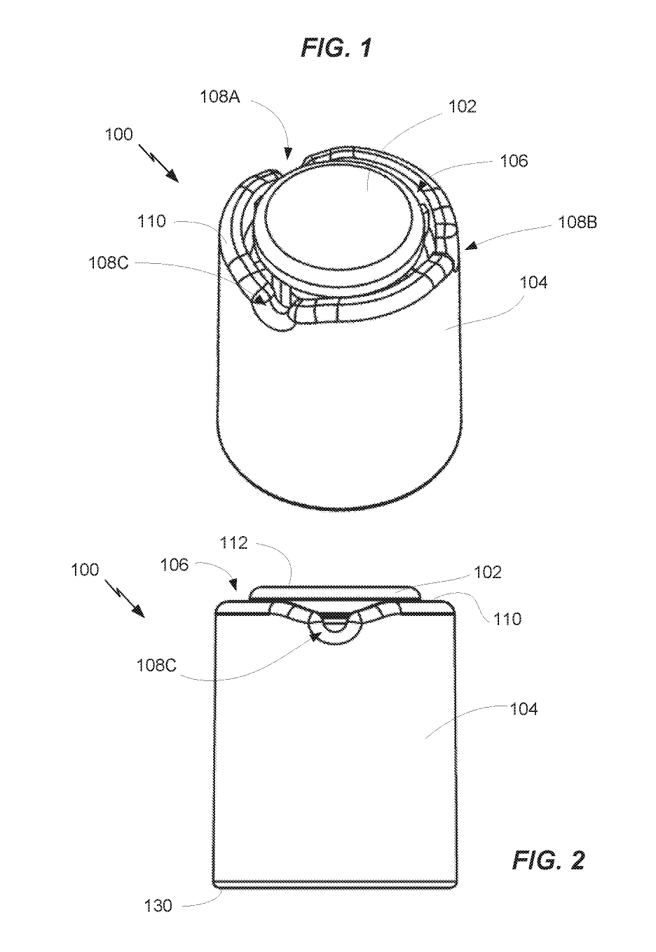

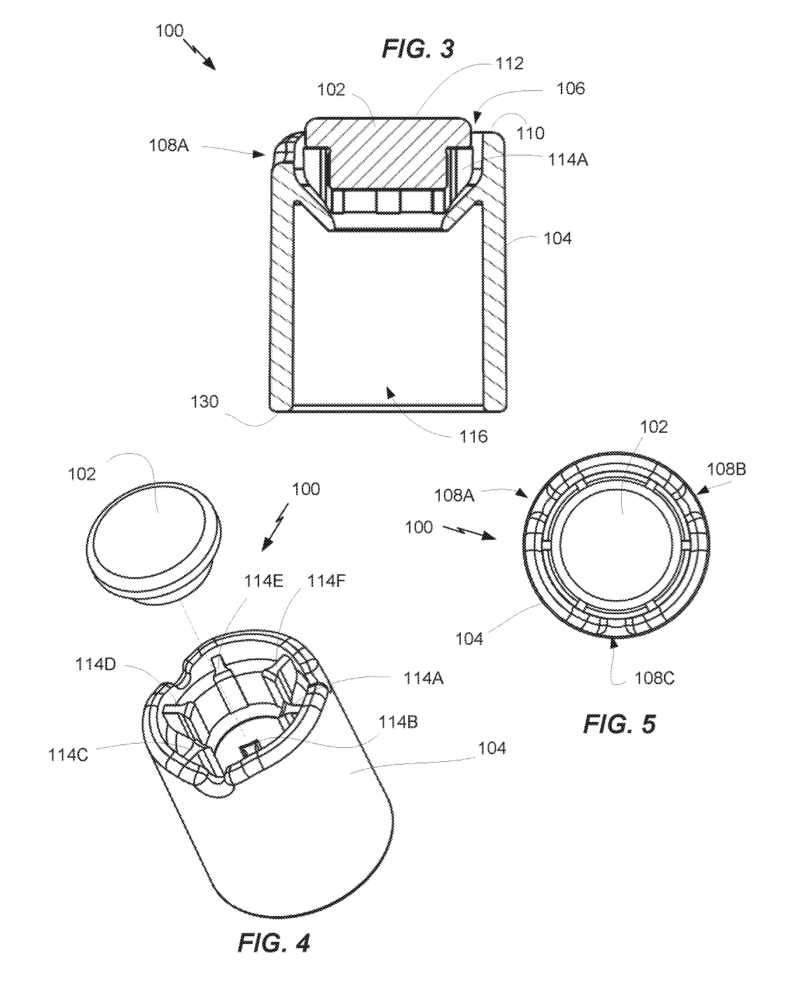

Turning in detail to the drawings, FIGS. 1-6 show various views of a vented microdermabrasion tip 100 with a fixed abrasive surface 112. The vented microdermabrasion tip 100 includes a disk-shaped abrasive component 102 coated or configured with a microdermabrasive surface 112 and fixed to a tubular sleeve 104 by a set of annular mounts 114A-E (FIG. 4). The sleeve 104 may surround an outer periphery of the abrasive component 102, so that a gap 106 exists between the abrasive component and the sleeve 104 leading to an internal air passage 116 through the sleeve 104 (FIG. 5). A distal surface 112 of the microdermabrasion tip 100 may be free of holes and may be coated with an abrasive material. The distal (working) surface 112 may extend beyond (e.g., be raised above) the distal end 110 of the sleeve 104. An end 130 of the mount opposite to the abrasive component 102 may be configured for coupling to a channel of a suction apparatus leading to an inlet of an air pump.

The sleeve 104 may be made of any suitable structural material, for example structural plastics and/or stainless steel. At least three ventilation holes 108A-C are evenly spaced around the periphery of the sleeve, as shown. The ventilation holes 108A-C are configured to freely admit air into the interior channel 116 even when the gap 106 is closed off by a user's skin around the entire periphery of the abrasive component 102. The depicted holes are shown at roughly 2× scale and are large enough to equalize pressure and maintain air flow, creating an opening below the distal end 110 of about 50 mm² each. While ventilation holes of other dimensions and configurations may also be useful, the illustrated dimensions should provide guidance for useful ventilation proportions. Vents should be large enough to prevent choking of the air pump but not so large that air velocity adjacent to the abrasive surface 112 is too slow to carry away abraded debris.

The abrasive component 102 may be made of a stainless steel or other suitable structural material, with a bonded layer of abrasive grit coating the working surface 112. For example, a diamond powder material may be bonded to the abrasive surface 112 at the distal end of tip 100. Bonding of the abrasive material may be by brazing, adhesion using an adhesive, or other method. Suitable abrasive materials may include, for example, ANSI Standard B74.18-1996 220 grit (66 μm) diamond or metal oxide powders. The range of useful grit sizes for microdermabrasion are expected to be within about 100 grit (148 μm) to about 240 grit (52 μm), depending on the age, skin type, and area of skin being treated. Users may need to try different abrasive grits to find the abrasive that works best for their intended use. Accordingly, the tip 100 may be configured so that the end user can remove the abrasive component 102 and replace it with a component of the same size but different abrasive characteristics. In addition, or in the alternative, additional tips may be provided in different shapes or sizes, with abrasive of the same or of different grit sizes.

In the illustrated embodiment, the abrasive component 102 is fabricated as a rigid body, meaning that it does not undergo any flexing or bending during its intended use that is perceivable by the end user. It should be appreciated, however, that the component 102 may be configured as a resilient member. An example of a resilient abrasive component is provided herein below, in connection with FIG. 15.

Advantageously, ventilation provided by the vents 108A-C relieves differences in static pressure between the interior channel 116 and the exterior of the sleeve 104, preventing the pressure difference from forcing the skin against or into the gap 106. At the same time, removal of abraded material continues without interruption even if the user's skin has choked off airflow through most or all of the gap 106. If choking occurs, the user does not feel any change in flow or pressure difference. Vents 108A-C should be sufficiently large and set away from the distal end 110 to prevent the buildup of a pressure difference even if the gap 106 is obstructed by skin. By experimenting with different variations in the shape, size and position of the ventilation holes and in the amount by which the abrasive surface 112 is elevated above the distal end 110, the inventors hereof have discovered the useful configuration that is illustrated in FIGS. 1-5. Other configurations may also be useful.

With reference to FIG. 5 (shown at roughly 2× scale), the component 102 has a diameter in the range of about 0.5-3 cm, for example, about 1.4 cm, with a beveled edge radius of about 1 mm. The distal working surface 112 is elevated about 1.5 mm past the distal end 110 of the sleeve 104, within a useful range of about 1-3 mm elevation. Each vent 108A-C is about 2-4 mm deep (as illustrated, about 2.5 mm deep) and about 2 mm in diameter at its lower end, tapering to a much larger size of about 8-12 mm where it meets the upper surface 110.

To use the microdermabrasion device, the user applies the abrasive tip against her skin with gentle hand pressure, and moves the tip over the skin surface in a generally elliptical motion while applying gentle hand pressure. Air flow provided by the air pump removes abraded material from around the abrasive component without causing skin to be drawn towards tip or any increase in contact between the abrasive surface 112 and the user's skin. Therefore, the skin does not stick to the tip, making it easier for the user to control the degree of abrasion by modulating hand pressure and to move the abrasive surface smoothly and continuously across the surface of the skin.

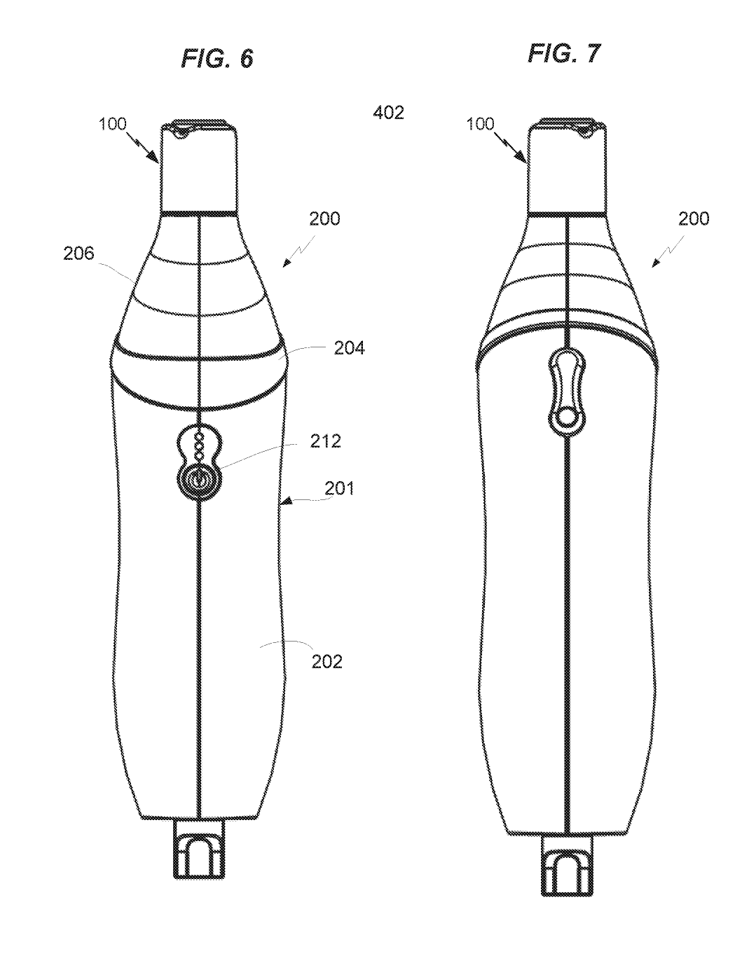

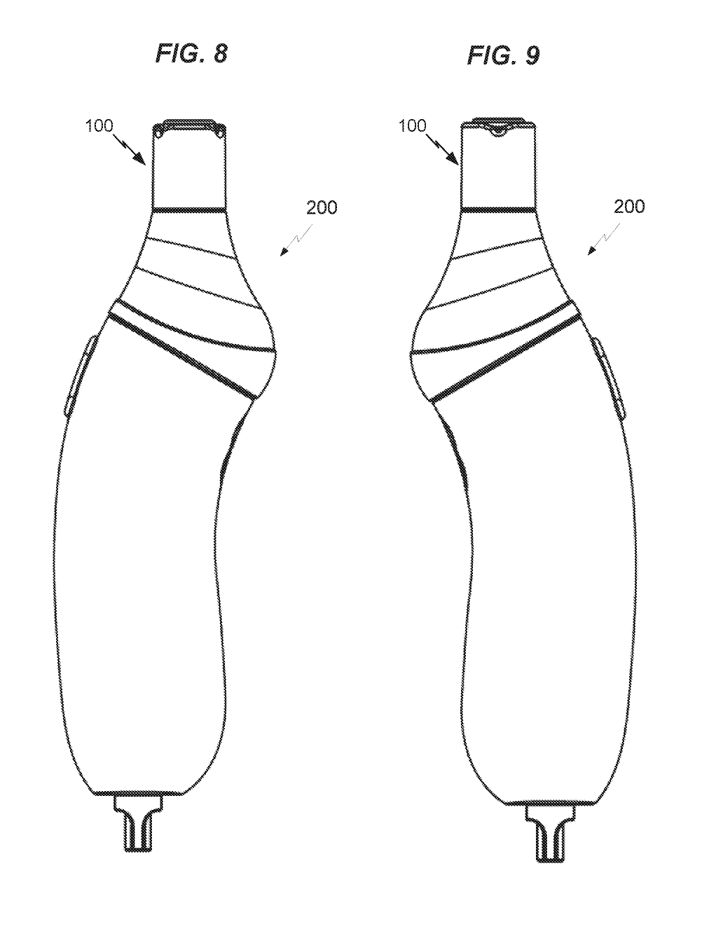

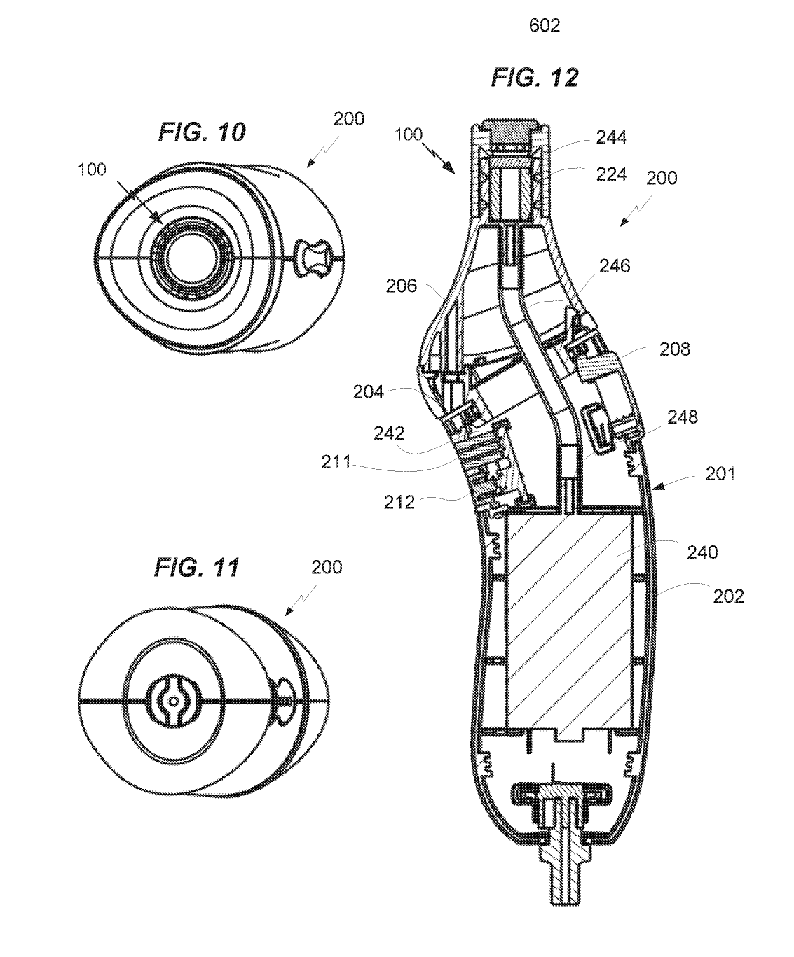

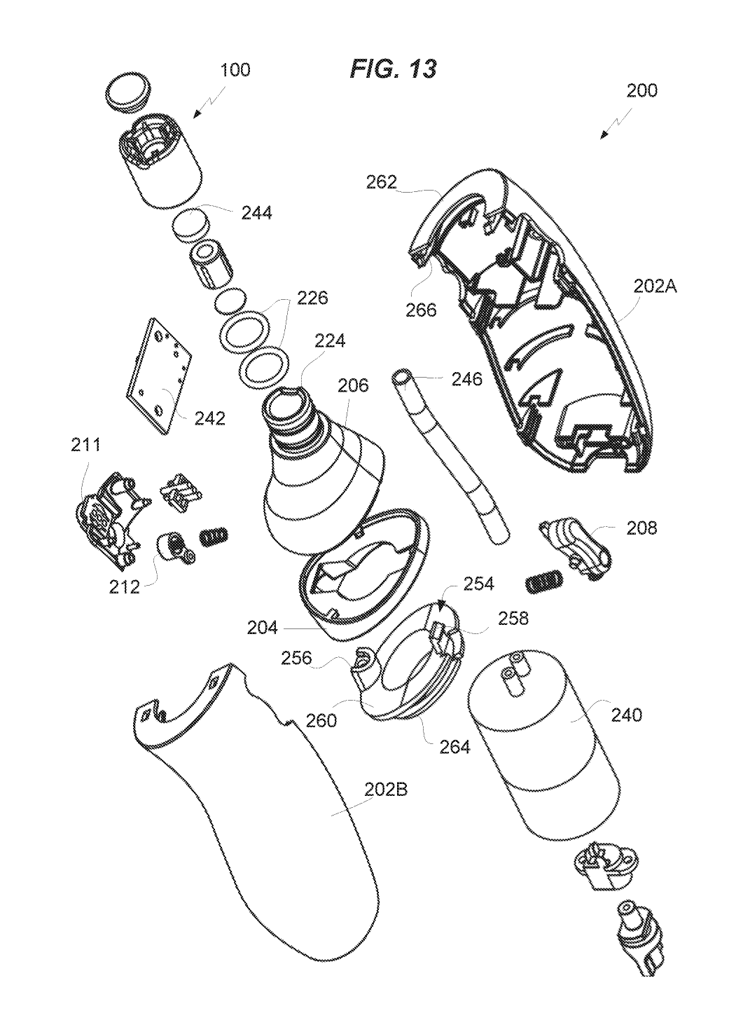

FIGS. 6-13 show various views of a portable handheld microdermabrasion assembly 200 including an ergonomically contoured handle 201 divided into at least two articulating sections 202, 206 separated by an annular cam ring 204. Each of the at least two articulating sections 202, 206 may be coupled to one another and rotatable (e.g., capable of swiveling) around a longitudinal axis of a longer one 202 of the at least two articulating sections, using any suitable mechanism (not shown), for example, respective interior sleeve bearings around a jointed shaft. Surprisingly, as the articulating sections rotate with respect to one another, the angle between them changes, thereby enabling angle adjustment between a lower one of the sections configured as a grip and an upper one of the sections supporting a microdermabrasion tip. In other words, the sections 202, 206 and spacer 204 may be configured with respect to the rotation mechanism such that rotation of the section 206 around the longitudinal axis of section 202 causes a distal end at tip 220 of the handle to be displaced from the longitudinal axis. Interior details regarding the illustrated mechanism for enabling rotation and angle adjustment between the two sections are described below in connection with FIGS. 12-13.

FIGS. 6-13 show the microdermabrasion device in a configuration wherein the distal end at the tip 220 is substantially aligned with the longitudinal axis of section 202. This alignment can most clearly be seen in FIG. 2. Note there is an offset of about one-half an average radius ‘R’ of section 202 between a central axis of the tip 220 and a central axis of section 202. Any offset between about zero to ‘R’ should be suitable.

The two sections 202, 206 may be locked into position relative to one another using a locking tab 208, as shown in FIG. 13. When engaged, the tab 208 prevents swiveling of the short handle section 206 relative to the long handle section 202. When disengaged, the sections 202, 206 are free to swivel with respect to one another along a path defined by the cam ring 204 and may lock into place at two or more points along the path, with or without a manually operated locking mechanism such as the locking tab. As used herein, to “swivel” means to rotate.

The handle 201 and its components may be made of any suitable structural material, for example structural plastics and/or stainless steel, and may include optional over-molded insets of a rubberized plastic or rubber material (not shown) to enhance grippability. In some embodiments, the over-molded insets may be omitted from the microdermabrasion assembly 200, as shown.

The microdermabrasion assembly 200 may include an air pump 240 (FIGS. 12, 13) disposed in one or both of the at least two articulating sections, e.g., longer section 202, configured to draw air through the distal end of the handle. Any suitable air pump may be used. It may be desirable to provide the air pump in a configuration that is not battery powered with a connector (not shown) to a wall outlet. Upper portions of a power connection for a wall outlet, including a connector 170 and strain relief bushing 172, are shown in FIGS. 12-13. The use of external power may enable the assembly 200 to provide more powerful and consistent suction through the tip 220 than may be possible using battery power. For example, the air pump may be configured to draw air through the channel at a rate of about 3.5 liters per minute, for the illustrated channel size. Battery power may also be used, if desired and suitably powerful.

Power to the air pump may be controlled using a mechanical switch or electronic switch 212 (FIGS. 6, 12) on an exterior of the handle 201. The switch 212 may be configured as a toggle coupled to a timer in electronic circuitry 242 (FIGS. 12, 13), such that, based on the switch 212 being toggled on, the power to the unit remains on for a definite period of time or indefinitely until the switch is toggled off. Timer-based pump control algorithms may be executed by a microprocessor in circuitry 242 coupled to a timer. A power-on event (e.g., a toggle-on signal) may initiate a timer or counter that counts up and checks the counter value against a constant or user-controlled threshold value, using a program loop. Once the counter reaches or exceeds the threshold value, the loop is terminated and initiates a “power-off” sequence. The power-off sequence may simply cut power or provide an audible or visible signal alerting the user for a predetermined warning period of time (e.g., 1-10 seconds) before power will be shut off, and then cut power if no user input is received during the warning period. If the user toggles the switch during the warning period, the termination sequence may reinitiate the timing loop.

Other control functions may be included, for example, to control the speed of the air pump 240 to different speeds between zero (off) and maximum speed, in response to user input. The electronic switch 212 may toggle between different pump speeds and power off, with indicator lights on an instrument panel 211 indicating a present speed setting. Pump speed or other operating parameters may optionally be indicated using a control panel of LED's or an LCD display screen. LED indicator lights, for example, are shown assembled to a switch mechanism 212 under the instrument panel 211, in FIG. 12.

A filter 244 (FIGS. 12, 13) may be interposed between the microdermabrasion tip 100 and the inlet of the air pump. In the alternative, or in addition, a filter may be interposed between an outlet of the air pump and an exterior of the handle 201. The filter should be designed to be easily removed and replaced. The filter material may be designed to be disposable or washable.

The microdermabrasion assembly 200 may further include a mount 224, shown in FIGS. 12 and 13. The mount 224 may be positioned at the distal end configured for mounting a microdermabrasion tip 100 around a channel leading to an inlet of the air pump 240. The mount 224 may include a cylindrical outer surface sized to slip inside a cylindrical inner surface of the microdermabrasion tip 100. The cylindrical outer surface of the mount 224 may include a double O-ring seal, for example dual O-rings 226 disposed in respective O-ring grooves around an outer circumference of the mount 224. The O-rings may provide a dual function by providing frictional resistance holding the microdermabrasion tip 100 temporarily in place and sealing the air channel to the air pump inlet, so that all of the suction provided by the air pump is directed to the microdermabrasion tip. The O-rings may provide additional benefits such as cost efficiency, simplicity, durability, and maintainability to the mount 224. Other mounting systems may be used if desired.

Rotating each of the at least two articulating sections with the tab 208 disengaged may cause the distal end of the handle to move in a substantially elliptical path having a first end substantially aligned with the longitudinal axis and a second end at a point of maximum displacement from the longitudinal axis. At the point of maximum displacement, an acute angle is formed between respective central longitudinal axes of the at least two articulating sections of not less than about 40°. Placing the assembly 200 in this position may facilitate self-treatment of dorsal or lateral portions of the body, may assist in alleviating wrist fatigue, or both.

Referring to FIGS. 12-13, internal aspects of the assembly 200 are shown in cross-section and exploded views, respectively. An inlet 248 of the air pump 240 is fluidly coupled to an outlet of the mount 224 by a flexible hose or tube 246. The air pump 240 may exhaust to an interior of the lower section 202, which section may be vented with suitable ventilation channels or through holes. Air may be drawn from the tip 100 through the filter 244 into the tube 246.

The swiveling and angle adjustment mechanism may include the upper section 202 slideably engaged with the cam ring 204 and lower section 202 by an engagement ring 254. The engagement ring 254 may be fixed to the upper section 202 by a threaded fastener (not shown) engaged at mounting bracket 256 and by clip 258 disposed around the engagement ring 254 approximately 180 degrees from the mounting bracket 256. The engagement ring 254 may be slideably engaged with the lower section 202 by a lower surface of the upper flange 260 held against an outer surface of exterior flange 262 integral to the lower section 202 by the upper surface of the lower flange 264 which is held against a lower surface of the lower flange 266 of section 202. The long section 202 may be assembled from mating halves 202A, 202B by snap engagement and/or threaded fasteners (not shown).

Hence, the engagement ring 254 may be inserted between the section 202 halves 202A, 202B in the manner shown and described during assembly, after being first fixed to the upper section 206 by the mounting bracket 256 and clip 258. The cam ring 204 may thereby be fixed between the engagement ring 254 and the upper section 206. The cam ring 204 is contoured as shown, thereby providing a desired amount of lateral displacement and angle adjustment between the sections 206, 202 when the upper section 206, cam ring 204 and engagement ring 254 are rotated as a body around the lower section 202. Different amounts of lateral displacement and angle adjustment with rotation may be provided for the assembly 200, by substituting a differently-contoured cam ring 204 in the assembly.

The rotation of the sections 206, 202 relative to one another may be in circular rotation defined by the interlocking, sliding relationship of the engagement ring flanges 260, 264 and lower section flanges 262, 266. The locking tab 208 may be engaged with a notch in the lower flange 264 to hold the two sections 202, 206 in a desired rotational position. Similar, additional notches in the flange 264, not visible in the illustrated views, may be provided around the flange 264 to enable locking the sections 202, 206 in place at other rotated positions. In an aspect, rotating each of the at least two articulating sections causes the distal end of the handle to move in a substantially elliptical path having a first end substantially aligned with the longitudinal axis and a second end at a point of maximum displacement from the longitudinal axis. When the distal end of the handle is located at the point of maximum displacement, an acute angle may be defined respective central longitudinal axes of the at least two articulating sections that is not less than about 40°.

In one or more exemplary designs, control functions of the described microdermabrasion device, for example pump control algorithms, may be implemented in hardware, software, firmware, or any combination thereof. If implemented in software, the functions may be stored as one or more instructions or code on a non-transitory computer-readable medium. Computer-readable media includes computer storage media or any other non-transitory tangible medium that facilitates holding a computer program in storage or machine memory. By way of example, and not limitation, such computer-readable media can comprise RAM, ROM, EEPROM, CDROM or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor. Combinations of the above should also be included within the scope of computer-readable media.

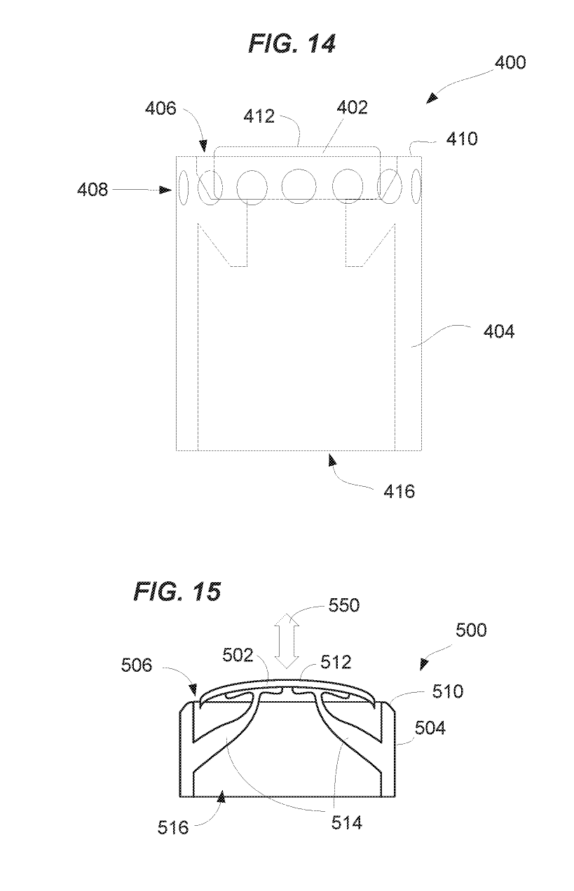

FIG. 14 shows an alternative vented microdermabrasion tip 400 illustrating the use of multiple vent holes 408 through a sleeve 404 into a main channel 416. The holes 408 (unlike vents 108A-C of tip 100) are unconnected to the distal end 410 of the sleeve 404. Air is drawn through the gap 406 and vents 408 through main channel 416 by a pump. The abrasive component 402 can be configured as described for tip 100. While FIG. 14 shows the distal abrasive surface 412 of the component 402 above the distal end 410 of the sleeve 404, it should be appreciated that the abrasive surface 412 can be level with or below the distal end 410 of the sleeve. In any of those configurations, the vent holes 408 maintain air flow through the channel 416 and balance static pressure between the inside of the sleeve 404 and its exterior even if the gap 406 is obstructed by the user's skin. Therefore the user does not feel the tip pulling on the skin and can move the abrasive surface smoothly and continuously over the skin without impediment or excessive abrasion.

FIG. 15 shows an alternative vented or unvented microdermabrasion tip 500 illustrating the use of a resilient abrasive component 502, in side cross section. The component 502 may be itself resilient, for example, it may comprise a generally convex outwards, arcuate, resilient sheet of stainless steel, laminate, or fiber composite material with an adhered abrasive material as previously described on its distal surface 512. The arcuate, slightly convex distal surface engages the skin under treatment without any abrupt discontinuities, facilitating a more comfortable and controllable application of the abrasive material. In an alternative, the component 502 may be of similar shape but substantially rigid, but may be supported by substantially resilient arms 514 that flex and allow the component 505 to move in the direction indicated by the arrow 550, as force is applied or removed in a direction parallel to the arrow 550. Likewise, if a resilient component 502 is used, the support arms 514 may be similarly resilient. In alternative embodiments, the support arms 514 may be substantially rigid, without noticeable flexure during use. While FIG. 15 shows cantilevered, resilient support arms, it should be appreciated that other types of resilient members, for example, coil springs, Belleville washers, or leaf springs, may be used to similar effect. The resilient member should be configured to respond to relatively light pressure (e.g., 0.5 to 5 pounds) with motion in the range of about 1 to 5 mm.

Vent holes (not shown) may be provided through the sleeve 504 as previously described, or may be omitted. Air is drawn through the annular gap 506 (and optionally, side vents if present) through main channel 516 by a pump. Vent holes (not shown) may be provided to maintain air flow through the channel 516 and balance static pressure between the inside of the sleeve 504 and its exterior even if the gap 506 is obstructed by the user's skin. In an alternative, vent holes may be omitted and instead, pressure control provided by the reaction of the flexible abrasive component 502 to applied pressure, whether applied by suction force or by hand. The flexibly supported component 502 moves in response to applied pressure having a component generally perpendicular to the component's 502 distal surface 512 and parallel to its principal direction of motion as indicated by the arrow 550. As pressure increases, the component 502 moves downwards. The movement of the abrasive component 502 may increase the flow capacity of the annular gap 506 and relieve static air pressure differential between the interior channel 516 and the exterior of the tip 500. Optionally, the downward motion may open or enlarge side air vents (not shown) in the sleeve 504, further relieving pressure differential. In addition, as the component 502 moves downwards, the rounded safety lip 510 advances towards the skin, gradually pushing the skin away from the abrasive surface 512, to prevent application of excessive pressure to the skin. Therefore, as the level of suction or hand pressure increases, the contact pressure between the skin and the abrasive surface decreases as the abrasive surface gradually retreats into the sleeve 504. The resiliency of the tip increases the comfort and safety of the microdermabrasion process by making it easier for the user to maintain a consistent pressure between the abrasive distal surface 512 and the surface of the skin under treatment, as the tip is moved over the skin. The user can more easily move the abrasive surface smoothly and continuously over the skin without impediment or excessive abrasion.

Having thus described embodiments of a microdermabrasion tip and method of use, it should also be appreciated that various modifications, adaptations, and alternative embodiments thereof may be made within the scope and spirit of the present invention. For example, specific types of microdermabrasion tip and specific handle contours been illustrated, but the inventive concepts described above would be equally applicable to implementations with other tips and handle contours.

1. A vented abrasive tip for microdermabrasion or exfoliation of skin with vacuum-assisted removal of debris, the tip comprising:
a tubular sleeve comprising a wall surrounding an internal air passage;
an abrasive component attached to the sleeve, the abrasive component having a distal abrasive surface centered over and extended beyond a distal end of the sleeve, so that a gap exists between the abrasive component and the sleeve leading to the internal air passage; and
an opening through the sleeve below its distal end for venting air to the internal air passage in the event the gap becomes obstructed during use.

2. The vented abrasive tip of claim 1, further comprising means for supporting the abrasive component in the tubular sleeve.

3. The vented abrasive tip of claim 1, wherein the means for supporting is non-resilient, such that the abrasive component is fixed in position.

4. The vented abrasive tip of claim 1, wherein the means for supporting is resilient, such that the abrasive component moves parallel to a longitudinal axis of the tubular sleeve in response to varying pressure of application of the abrasive component to skin during microdermabrasion or exfoliation.

5. The vented abrasive tip of claim 1, wherein the means for supporting is configured to move at least 1 mm and not more than 5 mm is response to compressive force applied to the abrasive component.

6. The vented abrasive tip of claim 1, further comprising a plurality of openings, the plurality including the opening as a like member, disposed around the tubular sleeve biased toward the distal end.

7. The vented abrasive tip of claim 6, wherein each of the plurality of openings opens via a channel to the distal end of the tubular sleeve and to the internal air passage.

8. The vented abrasive tip of claim 6, wherein each of the plurality of openings is closed to the distal end of the tubular sleeve and open to the internal air passage.

9. The vented abrasive tip of claim 1, wherein the distal abrasive surface is flat and disk-shaped.

10. The vented abrasive tip of claim 1, wherein the distal abrasive surface is convex and contoured smoothly from a center portion distal from the tubular sleeve to a perimeter proximal to the tubular sleeve.

11. An abrasive tip for microdermabrasion or exfoliation of skin with vacuum-assisted removal of debris, the tip comprising:
a tubular sleeve comprising a wall surrounding an internal air passage;
an abrasive component attached to the sleeve, the abrasive component having a distal abrasive surface centered over and extended beyond a distal end of the sleeve, so that a gap exists between the abrasive component and the sleeve leading to the internal air passage; and
a resilient member supporting the abrasive component in the tubular sleeve.

12. The abrasive tip of claim 11, wherein the resilient member is selected from the group consisting of cantilevered arms, coil springs, Belleville washers, or leaf springs.

13. The abrasive tip of claim 11, wherein the resilient member is configured to resiliently support the abrasive component through motion parallel to a longitudinal axis of the tubular sleeve in response to varying pressure of application of the abrasive component to skin during microdermabrasion or exfoliation.

14. The abrasive tip of claim 11, further comprising an opening through the sleeve below its distal end for venting air to the internal air passage.

15. The abrasive tip of claim 11, further comprising a plurality of openings, the plurality including the opening as a like member, disposed around the tubular sleeve biased toward the distal end.

16. The abrasive tip of claim 15, wherein each of the plurality of openings opens via a channel to the distal end of the tubular sleeve and to the internal air passage.

17. The abrasive tip of claim 15, wherein each of the plurality of openings is closed to the distal end of the tubular sleeve and open to the internal air passage.

18. The abrasive tip of claim 15, wherein the distal abrasive surface is flat and disk-shaped.

19. The abrasive tip of claim 15, wherein the distal abrasive surface is convex and contoured smoothly from a center portion distal from the tubular sleeve to a perimeter proximal to the tubular sleeve.

20. A portable apparatus for microdermabrasion or exfoliation of skin with vacuum-assisted removal of debris using an abrasive tip, the apparatus comprising:
a handle enclosing an air pump; and
an abrasive tip mounted to an end of the handle and coupled to the pump via a tube, the tip comprising:
a tubular sleeve comprising a wall surrounding an internal air passage;
an abrasive component attached to the sleeve, the abrasive component having a distal abrasive surface centered over and extended beyond a distal end of the sleeve, so that a gap exists between the abrasive component and the sleeve leading to the internal air passage; and
at least one of: an opening through the sleeve below its distal end for venting air to the internal air passage in the event the gap becomes obstructed during use, or a resilient member supporting the abrasive component in the tubular sleeve.
